(19) United States
(12) Patent Application Publication
(10) Pub. No.: US 2022/0125990 A1
(43) Pub. Date: Apr. 28, 2022

(54) SYSTEMS AND METHODS FOR HOT-ISOSTATIC PRESSING TO INCREASE NITROGEN CONTENT IN SILICON NITRIDE

(71) Applicant: SINTX Technologies, Inc., Salt Lake City, UT (US)

(72) Inventors: Bryan J. McEntire, Salt Lake City, UT (US); Bhajanjit Singh Bal, Salt Lake City, UT (US); Ryan M. Bock, Salt Lake City, UT (US)

(21) Appl. No.: 17/509,541

(22) Filed: Oct. 25, 2021

Related U.S. Application Data
(60) Provisional application No. 63/104,852, filed on Oct. 23, 2020.

(51) Int. Cl. A61L 27/10; C04B 35/593; C04B 35/645; A61L 27/54

(52) U.S. Cl. CPC A61L 27/10; C04B 35/5935; C04B 35/6455; A61L 27/105; C04B 2235/6567; C04B 2235/3873; C04B 2235/3217; C04B 2235/3225; C04B 2235/3895; A61L 27/54

(57) ABSTRACT

Methods and systems for manufacturing a ceramic or glass material component supersaturated in nitrogen are disclosed. The method for manufacturing a component typically comprises receiving the ceramic or glass material within a containment vessel; simultaneously heating and applying isostatic pressure to the ceramic or glass material within the containment vessel to a first temperature and a first pressure using pressurizing nitrogen gas; holding the first temperature and the first pressure for a period of time; cooling the ceramic or glass material within the containment vessel to a second temperature while maintaining the first pressure; and depressurizing the containment vessel to a second pressure.

### CROSS-REFERENCE TO RELATED APPLICATIONS

This application claims priority to U.S. Provisional Application No. 63/104,852 filed Oct. 23, 2020, the entirety of which is incorporated by reference herein.

### FIELD

The present disclosure relates to systems and methods for hot-isostatic pressing to increase the nitrogen content in ceramics and glasses. Aspects of the disclosure relate to components or implants produced by the systems and methods disclosed herein.

### BACKGROUND

The need for safe and reliable inactivation of viruses and lysis, of bacteria, and fungi is universal. There is a broad need to control the pathogens that affect human health and agricultural products. There is a need for improved and varied methods for manufacturing compounds, materials, or components that possess antipathogenic properties for biomedical implants, devices, and fomites.

### SUMMARY

The present disclosure relates to methods and systems for manufacturing a compound, material, or component, and particularly to manufacturing a compound, material, or component using hot-isostatic pressing with non-adiabatic cooling. Aspects of the disclosure also relate to compounds, materials, components or implants produced by the methods disclosed herein.

The methods for manufacturing a compound, material, component, or implant disclosed herein advantageously enable their saturation with nitrogen. In addition, the methods disclosed herein enable the production of compounds, materials, components or implants with increased antipathogenic properties due to the increased nitrogen content. The methods of manufacture utilize a unique process to produce compounds, materials, components or implants that simultaneously have increased density, supersaturation of nitrogen, and improved anti-pathogenicity, which is highly desirable. In some instances, the components may be configured to be implants supersaturated with nitrogen, which is desirable for prosthetic dental implants, spinal implants, total joint implants, and the like.

In accordance with a first aspect, a method for manufacturing a component comprising a ceramic or glass material typically includes placing the ceramic or glass compound, material, or component made from the ceramic or glass compound or material, within a containment vessel; simultaneously heating and applying isostatic pressure to the ceramic or glass compound, material, or component within the containment vessel to a first temperature and a first pressure using pressurizing nitrogen gas; holding the first temperature and the first pressure for a period of time; cooling the ceramic or glass material within the containment vessel to a second temperature while maintaining the first pressure; and depressurizing the containment vessel to a second pressure. The finished component comprises the ceramic or glass compound, material, or component supersaturated with nitrogen.

The method may further include removing the compound, material, or component from the containment vessel. The ceramic or glass material may be a powder and may include silicon nitride. In some aspects, the ceramic or glass material may be sintered prior to being received within the containment vessel and/or the component may be pre-formed into a useful shape prior to being received within the containment vessel.

In various embodiments, the first temperature may be about 1800° C., the second temperature may be less than 100° C., the first pressure may be about 200 MPa, the second pressure may be about atmospheric pressure, and the period of time may be about 2 hours.

In some aspects, the cooling step increases an average flexural strength of the component by 200-300 MPa as compared to a component produced using adiabatic cooling.

According to a second aspect, provided is an implant that is produced by a method, which includes preforming a ceramic or glass material into a useful shape, placing the preformed ceramic or glass material within a containment vessel; simultaneously heating and applying isostatic pressure to the preformed ceramic or glass material within the containment vessel to a first temperature and a first pressure using pressurizing nitrogen gas; holding the first temperature and the first pressure for a period of time; cooling the ceramic or glass material within the containment vessel to a second temperature while maintaining the first pressure; and depressurizing the containment vessel to a second pressure. In some aspects, the compound, material, component, or implant may be silicon nitride (Si₃N₄) with further additions of about 0.1 wt. % or more of sodium oxide (Na₂O), lithium oxide (Li₂O), potassium oxide (K₂O) magnesium oxide (MgO), aluminum oxide (Al₂O₃), yttrium oxide (Y₂O₃), ytterbium oxide (Yb₂O₃), lanthanum oxide (La₂O₃), strontium oxide (SrO), calcium oxide (CaO), silicon dioxide (SiO₂), zirconium oxide (ZrO₂), boron trioxide (B₂O₃), phosphorus pentoxide (P₂O₅) or combinations thereof.

Preferably, the compound, material, component or implant may be antipathogenic. For instance, the compound, material, component or implant may inhibit the proliferation of at least one of bacteria, fungi, and viruses.

In some aspects, the implant has an average flexural strength that is 200-300 MPa higher than an implant produced using adiabatic cooling.

### BRIEF DESCRIPTION OF THE DRAWINGS

The accompanying drawings, which are incorporated in and constitute a part of this specification, illustrate several embodiments of the invention and together with the description serve to explain the principles of the invention.

FIG. 1 is a flow chart representation of an exemplary, non-limiting embodiment of a method for manufacturing a component in accordance with an aspect of the present disclosure.

FIGS. 2A-2D shows confocal laser scanning microscopy images and intensity maps for surfaces of specimens from a component manufactured without HIP, a component manufactured with an adiabatic cooling procedure, a component manufactured with a 10 ksi cooling procedure, and a component manufactured with a 25 ksi cooling procedure.

FIGS. 3A-3D shows low magnification secondary electron images of as-fired & as-pressed surfaces of Si₃N₄ samples subjected to sinter only, an adiabatic cooling procedure, a 10 ksi cooling procedure, and a 25 ksi cooling procedure.

FIGS. 4A-4D shows high magnification secondary electron images of as-fired & as-pressed surfaces of Si₃N₄ samples subjected to sinter only, an adiabatic cooling procedure, a 10 ksi cooling procedure, and a 25 ksi cooling procedure.

FIGS. 5A-5D shows back-scattered electron images of polished cross-sections of Si₃N₄ samples subjected to sinter only, an adiabatic cooling procedure, a 10 ksi cooling procedure, and a 25 ksi cooling procedure.

FIG. 6 shows x-ray diffraction patterns for Si₃N₄ samples subjected to sinter only, an adiabatic cooling procedure, a 10 ksi cooling procedure, and a 25 ksi cooling procedure. A β-Si₃N₄ standard is included for reference.

FIG. 7 shows the results of a biofilm assay using S. epidermis on four ceramic materials manufactured through different processes.

FIGS. 8A-8D show laser scanning micrographs of Si₃N₄ samples subjected to sinter only, an adiabatic cooling procedure, a 10 ksi cooling procedure, and a 25 ksi cooling procedure. FIG. 8E shows the volume of deposited mineralized bone matrix measured by scanning laser microscopy. FIG. 8F shows the optical density value of medium at 450 nm following Alizarin red staining.

FIG. 9 shows Raman image maps and averaged Raman spectra for Si₃N₄ samples subjected to sinter only, an adiabatic cooling procedure, a 10 ksi cooling procedure, and a 25 ksi cooling procedure.

FIGS. 10A-10C shows the inorganic to organic ratio, the HAp crystallinity, and the HAp to TCP ratio for Si₃N₄ samples subjected to sinter only, an adiabatic cooling procedure, a 10 ksi cooling procedure, and a 25 ksi cooling procedure.

FIG. 11 shows the Weibull failure probability plot for silicon nitride lots processed with standard adiabatic HIP cooling and the experimental 25 ksi HIP cool cycle. Linear fits are included to generate Weibull distribution parameters.

FIGS. 12A-12C shows visible light stereomicrographs of fracture origins in specimens that exhibited strengths lower than 800 MPa. Arrows indicate flows at the fracture origins.

FIGS. 13A-13D shows the x-ray diffraction peak positions for [110], [210], [200], and [101] planes in β-Si₃N₄ samples subjected to sinter only, an adiabatic cooling procedure, a 10 ksi cooling procedure, and a 25 ksi cooling procedure.

It should be understood that the various aspects are not limited to the arrangements shown in the drawings.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Thus, references to one or an embodiment in the present disclosure can be references to the same embodiment or any embodiment; and, such references mean at least one of the embodiments.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, the term “silicon nitride” includes α-Si₃N₄, β-Si₃N₄, SiYAlON, SiYON, SiAlON, or combinations thereof.

As used herein, the term “component” includes the ceramic or glass material, a compound, an implant, a device, or similar, that is useful for antipathogenic purposes.

Generally, the ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc. Other than in the operating examples, or where otherwise indicated, all numbers expressing quantities of ingredients and/or reaction conditions may be modified in all instances by the term “about,” meaning within +/−5% of the indicated number.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Aspects of the present disclosure relates to systems and methods for manufacturing a component, and particularly to manufacturing a component using hot-isostatic pressing with non-adiabatic cooling. Under the high temperature and pressure conditions of hot isostatic pressing, the intergranular phase of the component is a liquid. In some examples, the intergranular phase liquid may have a consistency similar to melted container glass. Without being bound by theory, the solubility of nitrogen gas is high at the increased temperature and pressure of hot isostatic pressing; thus, by maintaining a high overpressure during cooling, the ability of nitrogen to precipitate out of the glass due to its decreased solubility is hindered. Also without being bound by theory, higher overpressures held for longer periods of cooling maintain a longer duration of nitrogen dissolution, thus increasing the supersaturation of nitrogen in the finished component. Even if overpressure is released too early, a significant amount of nitrogen may still be mobile within the intergranular phase of the component.

Methods disclosed herein may include gas-pressure hot-isostatic pressing to supersaturate glass-based compositions with nitrogen by using non-adiabatic cooling (i.e. holding the hot-isostatic pressing pressure constant during the cooling cycle). For example, the method may include holding a high and constant pressure of nitrogen in the hot-isostatic pressing operation during the entire cooling cycle. Surprisingly, this maintains the supersaturation of nitrogen within the hot-isostatic pressed material. The excess nitrogen is then available for antipathogenic purposes.

The methods for manufacturing a component disclosed herein advantageously enable the production of components supersaturated with nitrogen to increase the antipathogenic properties of the component. For example, the methods disclosed herein enable the production of antipathogenic components, such as biomedical implants and devices including but not limited to dental implants, spinal implants, joint components, orthopedic implants, pedicle screws, in-dwelling catheters, endotracheal tubes, colonoscopy scopes, and other similar devices and the like.

Alternatively, in some embodiments, the components may be manufactured as fomites, having a high contact surface. Non-limiting examples include materials or surfaces which are likely to carry infection, such as handles, knobs, trays, counters, furniture, levers, bed rails, chairs, moveable lamps, light switches, cellular phone cases, tray tables, small counter surfaces, or other surfaces, masks, cloth, drapes, gowns, and other clothes, or utensils, tools, instruments, fixtures, or the like. One of ordinary skill in the art would recognize other benefits to employing aspects of the instant invention in various industries.

Without being limited to a particular theory, increased nitrogen content within silicon nitride may provide a surface chemistry such that increased ammonia (NH₃) is available for the inactivation or lysis of virus, bacteria, or fungi.

Nitrogen elutes faster (within minutes) than silicon because surface silanols are relatively stable. For viruses, it was surprisingly found that silicon nitride may provide for RNA cleavage via alkaline transesterification which leads to loss in genome integrity and virus inactivation. Increasing the nitrogen content in a silicon nitride material ensures that nitrogen is available for the RNA cleavage to take place.

In an embodiment, the antipathogenic component may exhibit elution kinetics that show: (i) a slow but continuous elution of ammonia from the solid state rather than from the usual gas state; (ii) no damage or negative effect to mammalian cells; and (iii) an intelligent elution increasing with decreasing pH.

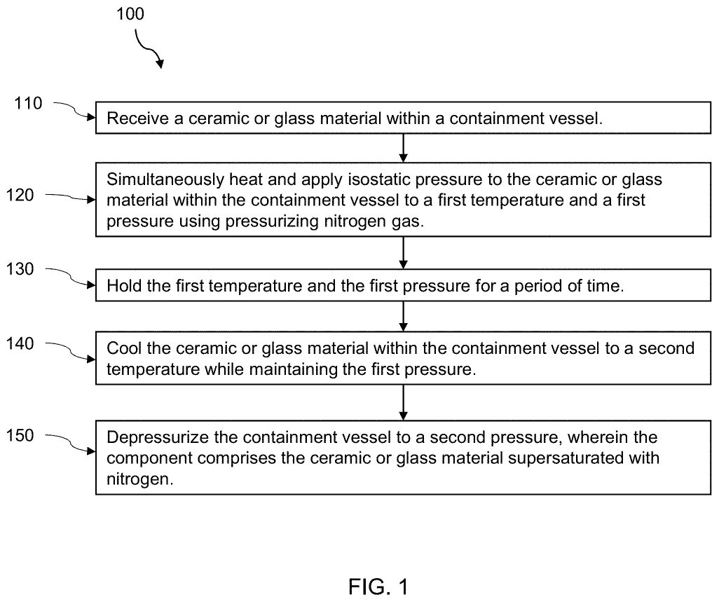

FIG. 1 is a flow chart of an exemplary, non-limiting method 100 for manufacturing a component comprising a ceramic or glass material. As a brief overview, method 100 includes receiving the ceramic or glass material within a containment vessel in step 110; simultaneously heating and applying isostatic pressure to the ceramic or glass material within the containment vessel to a first temperature and a first pressure using pressurizing nitrogen gas in step 120; holding the first temperature and the first pressure for a period of time in step 130; cooling the ceramic or glass material within the containment vessel to a second temperature while maintaining the first pressure in step 140, and depressurizing the containment vessel to a second pressure in step 150. The component formed by the method 100 comprises the ceramic or glass material supersaturated with nitrogen.

In step 110, a ceramic or glass material is received within a containment vessel. In some examples, the ceramic or glass material may include but is not limited to silicon nitride, glass ceramics, and/or polycrystalline ceramics. The ceramic or glass material may be in the form of a powder or may be pre-sintered prior to being placed in the containment vessel. The ceramic or glass material may already be formed into the shape of the desired component before being received within the containment vessel. Alternatively, the ceramic or glass material may be a powder when placed in the containment vessel and subsequently formed into the shape of the desired component within the containment vessel.

The ceramic or glass material may contain about 75 to about 99.9 wt. % of silicon nitride powder. For instance, the amount of silicon nitride powder present in the ceramic or glass material may be about 75 to about 80 wt. %, about 80 to about 85 wt. %, about 85 to about 90 wt. %, about 90 to about 95 wt. %; about 95 to about 99 wt. %, or about 100 wt. %, based on the total weight of the ceramic or glass material. The ceramic or glass material may be about 30 to about 99.9 wt. % of the component. For instance, the amount of ceramic or glass material present in the finished component may be about 30 to about 50 wt. %, about 50 to about 75 wt. %, about 75 to about 80 wt. %, about 80 to about 85 wt. %, about 85 to about 90 wt. %, about 90 to about 95 wt. %; about 95 to about 99 wt. %, or about 100 wt. %, based on the total weight of the component.

The method may employ a ceramic or glass material that includes about 25 wt. % or less of an additional powder, based on the total volume of the ceramic or glass material. In some instances, the amount of additional powder present in the ceramic or glass material is about 25 wt. % or less, about 20 wt. % or less, about 18 wt. % or less, about 16 wt. % or less, about 14 wt. % or less, about 12 wt. % or less, about 10 wt. % or less, about 8 wt. % or less, about 6 wt. % or less, about 4 wt. % or less, about 2 wt. % or less, or about 1 wt. % or less. In at least one instance, the ceramic or glass material consists of or consists essentially of silicon nitride powder and impurities. The additional powder may comprise about 0.1 wt. % or more of sodium oxide (Na₂O), lithium oxide (Li₂O), potassium oxide (K₂O) magnesium oxide (MgO), aluminum oxide (Al₂O₃), yttrium oxide (Y₂O₃), ytterbium oxide (Yb₂O₃), lanthanum oxide (La₂O₃), strontium oxide (SrO), calcium oxide (CaO), silicon dioxide (SiO₂), zirconium oxide (ZrO₂), boron trioxide (B₂O₃), phosphorus pentoxide (P₂O₅) or combinations thereof.

In step 120, heat and isostatic pressure are simultaneously applied to the ceramic or glass material within the containment vessel. The ceramic or glass material is heated to a first temperature and the pressure is increased to a first pressure. The temperature and pressure may be applied uniformly within the containment vessel. The containment vessel may be configured to operate at high temperatures and pressures during operation. In an embodiment, the containment vessel may use pressurizing nitrogen (N₂) gas to increase the pressure within the containment vessel.

In some embodiments, the first temperature may be about 1400° C. to about 1800° C. For example, the first temperature may be about 1400° C. to about 1450° C., about 1450° C. to about 1500° C., about 1500° C. to about 1550° C., about 1550° C. to about 1600° C., about 1600° C. to about 1650° C., about 1650° C. to about 1700° C., about 1700° C. to about 1750° C., or about 1750° C. to about 1800° C. In some examples, the first temperature may be at least 1600° C., at least 1700° C., or at least 1800° C. In one embodiment, the first temperature is about 1800° C. The containment vessel may be heated at a rate of about 10° C./minute until the first temperature is reached.

In an embodiment, the first pressure may be about 100 MPa to about 300 MPa. For example, the first pressure may be about 100 MPa to about 150 MPa, about 150 MPa to about 200 MPa, about 200 MPa to about 250 MPa, or about 250 MPa to about 300 MPa. In some examples, the first pressure may be at least 100 MPa, at least 150 MPa, at least 200 MPa, at least 250 MPa, or at least 300 MPa. In one embodiment, the first pressure is about 150 MPa. In another embodiment, the pressure increases by about 1.2 MPa per minute.

In step 130, the first temperature and the first pressure are held for a period of time. The period of time may range from about 0.5 hours to about 2 hours. For example, the first temperature and first pressure may be maintained within the containment vessel for at least about 0.5 hours, at least about 1 hour, at least about 1.5 hours, or at least about 2 hours. In one embodiment, the first temperature and first pressure may be maintained within the containment vessel for about 1 hour.

In step 140, the ceramic or glass material is cooled within the containment vessel to a second temperature while maintaining the first pressure (e.g. non-adiabatic cooling). The second temperature may be any temperature cooler than the first temperature. For example, the second temperature may be about 30° C. to about 50° C., about 50° C. to about 100° C., about 70° C. to about 120° C., or about 100° C. to about 150° C. In some examples, the second temperature may be less than about 100° C., less than about 50° C., or less than about 30° C. Without being limited to any one theory, the non-adiabatic cooling under the pressure of nitrogen gas allows for the ceramic or glass material to be supersaturated with nitrogen. In some embodiments, the ceramic or glass material may be cooled at a rate of between about 5° C. to about 10° C.

Supersaturation is dependent upon the composition of the material. The ceramic or glass material may be supersaturated in a range of 10 wt. % to 15 wt. %. In an embodiment, the ceramic or glass material includes at least about 1 wt. %, at least about 2 wt. %, at least about 3 wt. %, at least about 4 wt. %, at least about 5 wt. %, at least about 6 wt. %, at least about 7 wt. %, at least about 8 wt. %, at least about 9 wt. %, at least about 10 wt. %, at least about 11 wt. %, at least about 12 wt. %, at least about 13 wt. %, at least about 14 wt. %, or at least about 15 wt. % more nitrogen content than the ceramic or glass material that is not cooled non-adiabatically.

In step 150, the containment vessel is depressurized to a second pressure. In an embodiment, the second pressure may be about 0.1 MPa to about 5 MPa. For example, the second pressure may be about 0.1 MPa to about 0.5 MPa, about 1 MPa to about 2 MPa, about 2 MPa to about 3 MPa, about 3 MPa to about 4 MPa, or about 4 MPa to about 5 MPa. In one embodiment, the second pressure is about atmospheric pressure. In another embodiment, the pressure may be lowered at a rate of between about 2 MPa to about 3 MPa.

In some cases, method 100 may further include removing the component from the containment vessel. After the ceramic or glass material is cooled non-adiabatically and then the pressure is reduced, the component made of the ceramic or glass material supersaturated in nitrogen may be removed from the containment vessel.

According to a second aspect, provided is a component (e.g., an implant) comprising a silicon nitride supersaturated in nitrogen that is produced by a method including receiving the ceramic or glass material within a containment vessel; simultaneously heating and applying isostatic pressure to the ceramic or glass material within the containment vessel to a first temperature and a first pressure using pressurizing nitrogen gas; holding the first temperature and the first pressure for a period of time; cooling the ceramic or glass material within the containment vessel to a second temperature while maintaining the first pressure; and depressurizing the containment vessel to a second pressure. In some instances, the implant may be manufactured using one or more features of method 100, which is discussed above.

The component typically includes about 30 wt. % to about 100 wt. % ceramic or glass material. For instance, the amount of ceramic or glass material present in the finished component may be about 30 to about 50 wt. %, about 50 to about 75 wt. %, about 75 to about 80 wt. %, about 80 to about 85 wt. %, about 85 to about 90 wt. %, about 90 to about 95 wt. %; about 95 to about 99 wt. %, or about 100 wt. %, based on the total weight of the component. In various embodiments, the ceramic or glass material is silicon nitride, a glass ceramic, and/or a polycrystalline ceramic. The ceramic or glass material may contain about 75 to about 99.9 wt. % of silicon nitride powder. For instance, the amount of silicon nitride powder present in the ceramic or glass material may be about 75 to about 80 wt. %, about 80 to about 85 wt. %, about 85 to about 90 wt. %, about 90 to about 95 wt. %; about 95 to about 99 wt. %, or about 100 wt. %, based on the total weight of the ceramic or glass material. In an embodiment, the ceramic or glass material includes at least about 1 wt. %, at least about 2 wt. %, at least about 3 wt. %, at least about 4 wt. %, at least about 5 wt. %, at least about 6 wt. %, at least about 7 wt. %, at least about 8 wt. %, at least about 9 wt. %, at least about 10 wt. %, at least about 11 wt. %, at least about 12 wt. %, at least about 13 wt. %, at least about 14 wt. %, or at least about 15 wt. % more nitrogen content than the ceramic or glass material that is not cooled non-adiabatically.

Preferably, the component (e.g., an implant) is antipathogenic. For example, the component may inhibit the proliferation of at least one of bacteria, fungi, and viruses. In some examples, the bacteria may be S. epidermis. Additionally, and/or alternatively, the component may be configured to be an implant that enhances osteoblast cell proliferation. In at least one embodiment, the osteoblast cell proliferation increases on the implant as compared to an implant without the silicon nitride powder. The component may have a surface chemistry that accelerates bone repair. In some embodiments, the component (e.g., an implant) releases silicic acid and nitrogen from the surface of the component, which enhances the osteogenic activity of osteosarcoma and mesenchymal cells both at the initial stages of cell differentiation and during subsequent bony apatite deposition. Without being limited to any particular theory, the silicon nitride powder may stimulate the synthesis by osteoblasts of high-quality bone tissue, the former favoring bone matrix mineralization and the latter enhancing cell proliferation and formation of bone matrix. In addition, the component may possess a surface chemistry that is biocompatible and provides a number of biomedical applications including concurrent osteogenesis, osteoinduction, osteoconduction, and bacteriostasis.

The component may be in the form of an implant or device, which may be implanted in a patient's body in an area contacting or near bone. Non-limiting examples of implants include an intervertebral spinal spacers or cages, bone screws, orthopedic plates, and other fixation devices, articulation devices in the spine, hip, knee, shoulder, ankle, and phalanges, implants for facial or other reconstructive plastic surgery, middle ear implants, dental devices, pedicle screws, in-dwelling catheters, endotracheal tubes, colonoscopy scopes, and other similar devices and the like.

### EXAMPLES

### Example 1

It was hypothesized that holding pressure during cooling after hot isostatic pressing (HIP) could be used as a means to supersaturate Si₃N₄'s secondary silicon yttrium aluminum oxynitride (SiYAlON) glassy phase with excess nitrogen. It was further hypothesized that the excess nitrogen stored in the glass would cause accelerated release of ammonia into the physiologic medium as the SiYAlON was hydrolyzed, in turn enhancing the observed antimicrobial properties of the material. Four lots of silicon nitride test discs were processed from the same standard powder lot and subjected to different HIP cycles: (1) no HIP/sinter only at one atmosphere, (2) standard HIP with adiabatic (simultaneous release of pressure & temperature) cooldown, (3) 10 ksi hold during HIP cooldown, and (4) 25 ksi hold during HIP cooldown. Discs were subjected to a battery of materials characterization techniques and in vitro challenges with a biofilm-forming strain of S. epidermidis and pre-osteoblast KUSA-A1 mesenchymal stem cells in order to assess what effects, if any, the alteration of the HIP cycle had on the material's properties and biologic response.

It was also hypothesized that excess nitrogen sequestered into the minority SIYAlON glassy phase by controlling HIP pressure during cooling could (1) prevent or mitigate the formation of strength-limiting pores during HIP cooldown and (2) create a residual stress, measurable as a β-Si₃N₄ lattice strain, following processing. Mitigation of the gas-formed pore flaw population and the presence of a residual stress could act in tandem to inhibit crack propagation in the material leading to an observable increase in flexural strength. Flexural bar lots were made using adiabatic HIP cooling and 25 ksi hold during HIP cooling. Following flexural testing, the results from these lots were analyzed and compared to determine effects upon the material's strength distribution. Discs processed with differing HIP conditions as described above were subjected to X-ray diffraction. Peak positions of four principal peaks were compared for representative samples to assess lattice strain.

### Sample Size

Sample sizes for bacterial experiments were n=3 (bacteria) and n=variable per analysis technique (KUSA-A1 experiments) per condition and timepoint per established protocols. Sample sizes for flexural strength tests were n≥30 per ASTM C1161-186.

### Hypothesis/Acceptance Criteria

Null Hypothesis 1—Change in Antibacterial Properties: Controlling pressure during HIP cooldown will not modify Si₃N₄'s antibacterial properties such that CFU counts for a 25 ksi HIP cool sample would be lower than a sinter only or adiabatically cooled Si₃N₄ tested in parallel using SINTX's standard bacterial biofilm assay with a biofilm forming strain of Staphylococcus epidermidis (S. epidermidis). This hypothesis will be rejected if the CFU counts at 24 and 48 hour time points are lower with statistically significant p-values via a Student's t-test (2-tail, heteroscedastic) for the 25 ksi HIP cooldown material relative to the adiabatic cool material and the sinter only material.

Null Hypothesis 2—Strength Improvement: Holding pressure constant during HIP cooldown will not lead to improvement in the material's flexural strength. This hypothesis will be rejected if the average three point bending strength (per 940006) of the 25 ksi HIP cool material improves and comparison of the adiabatic cool test group and 25 ksi test group strength distributions yield a p-value less than 0.05 via a Student's t-test (2-tail, heteroscedastic, 95% confidence).

Null Hypothesis 3—Lattice Strain: Holding pressure constant during HIP cooldown will not impart residual lattice strain. This hypothesis will be rejected if X-ray diffraction peak shifts indicate a progressive increase in β-Si₃N₄ lattice strain (measured as a change in d-spacing) that correlates with magnitude of pressure applied during HIP cooldown.

### Materials and Methods

The silicon nitride powder lot used in this work had a nominal composition (mass percent) of 0.75% TiO₂ (New Brunswick, N.J. USA), 3.97% Al₂O₃ (UFX-MAR, Baikowski-Malakoff, Malakoff, Tex. USA), 5.96% Y203 (Triebacher Industrie AG, Althofen, Austria), and the balance as Si₃N₄ (Ube SN-E10, Ube, Japan). Briefly, this material was prepared by batching an aqueous slurry containing common commercially available organic dispersants and binders, milling the slurry in a circulating attrition mill (Q6, Union Process, Akron, Ohio USA) to deagglomerate the slurry, and then atomizing and drying the slurry using a spray dryer (NIRO® SD-6.3-N, GEA, Copenhagen, Denmark). Following spray drying, a common press lubricant was mixed into the powder at very low concentration (<1% by mass), the powder was then sieved to remove coarse agglomerates, and the powder was finally held in a controlled humidity chamber (Lunaire CEO 932, Tenney Environmental, New Columbia, Pa. USA) for at least one week to allow for stabilization of its residual moisture content.

All test discs were pressed to a target pressure of 300 MPa using a uniaxial hydraulic press (Carver, Wabash, Ind. USA) and a laboratory KBr pellet (∅12.7 mm×˜1 mm thick) die (VWR, Radnor, Pa. USA) per PARs 2096, 2097, 2098, & 2099 and co-fired using identical parameters except for their respective HIP cycles. The common firing steps were (1) binder removal in air up to 500° C. (ThermalTek, Concord, N.C. USA), (2) Binder removal and pre-sinter in 2 psi of nitrogen with a two hour hold at 1600° C. in a batch furnace (Centorr Vacuum Industries, Nashua, N.H. USA), and (3) sintering at 1710° C. for three hours in a continuous belt furnace (Centorr Vacuum Industries, Nashua, N.H. USA) under flood nitrogen (one atmosphere) conditions. The HIP (Quintus Technologies, LLC, Columbus, Ohio USA) cycles employed are detailed in Table 1.

TABLE 1

| PAR | Dwell Condition | Cooldown Condition |
|---|---|---|
| 2096 | N/A | N/A |
| 2097 | 1690° C., 2 hr, 22 ksi N₂ | Adiabatic cooldown |
| | | (Simultaneous temperature & pressure |
| | | release) |
| 2098 | 1690° C., 2 hr, 22 ksi N₂ | Ramp to 10 ksi, then hold at 10 ksi until |
| | | cooled to 300° C. |
| 2099 | 1690° C., 2 hr, 22 ksi N₂ | Ramp to 25 ksi and hold during cooling |
| | | until 300° C. is reached |

Following thermal processing, discs were subjected to CO₂ blasting, ultrasonic cleaning, and clean firing (700° C. in air for 30 minutes) to ensure their surfaces would be representative of processed implants.

Poly(ether ether ketone) (PEEK) discs were also produced for use as controls in bacterial biofilm assays. PEEK rod stock (∅12.7 mm) (ASTM D6262, Ketron® PEEK 1000, McMaster-Carr, Santa Fe Springs, Calif.) was machined by a commercial machining house into discs with a 1 mm thickness.

Flexural Bar Production: A control group of flexural bars processed with the standard adiabatic cool HIP cycle were produced. These test bars were dry isostatically pressed (310 MPa) in an elastomer mold to form square cross-section bar stock ˜21 mm×21 mm. The pressed bar stock was subjected to (1) binder removal and pre-sinter (Batch Furnace) by conducting a series of low temperature ramps and holds under vacuum up to 700° C. to volatilize organic constituents and then ramping to 1600° C. in 2 psi of nitrogen and holding for two hours, (2) sintering at 1710° C. for three hours (Belt Furnace) in flood nitrogen (one atmosphere) conditions, and (3) HIP cycle −1690° C., 2 hr exposure in 22 ksi N₂ with adiabatic cooldown. Following HIP, stock pieces were subjected to Archimedean density testing (n=3), then shipped to a commercial grinding house for processing into ASTM C1161-187 configuration B test bars (3 mm×4 mm cross-section×45 mm length). Once the ground bars were received back, their tensile faces were polished using a lapping machine (Lapmaster 15, Lapmaster, Mt. Prospect, Ill. USA).

The experimental flexural bar group was processed. The processing for this group was identical in all respects to that of the control group with the exception of the HIP cycle. This cycle consists of the same ramp and hold at 1690° for 2 hours at 22 ksi N₂. Instead of adiabatic cooling, the pressure ramps to 25 ksi N₂ during cooldown and holds until the vessel temperature is below 300° C. Post-HIP processing and testing were identical to what was performed for the control group.

Dimensional density measurements were carried out on six test discs. Diametral measurements were used to calculate linear shrinkage (S_{L}) from six test discs.

Cross-sectioned samples were prepared by mounting disc samples in epoxy resin (EpoThin, Buehler, Lake Bluff, Ill.), grinding several mm deep into the disc using a 400 grit diamond-embedded wheel on a surface grinder (ACC 12-24 DX Grind-X, Okamoto Corp., Vernon Hills, Ill. USA), then lapping using successively finer diamond grits (Engis, Wheeling, Ill. USA) against cast iron plates and culminating with a chemical mechanical polishing (CMP) process using colloidal silica (Engis) against a polishing pad (Suba IV, Engis).

Evaluations of Surface Morphology and Microstructure: As-fired surfaces were evaluated using a Confocal Laser Scanning Microscope (CLSM) (LEXT OLS5000, Olympus, Japan) to quantify their roughness parameters. As-fired and polished cross-sections prepared as described above were evaluated using a field emission gun scanning electron microscope (FEG-SEM) (Quanta, FEI, Hillsboro, Oreg. USA) equipped with an energy dispersive X-ray spectrometer (EDS) (EDAX, Mahwah, N.J. USA). Samples were sputter coated (PECS 1, Gatan, Pleasanton, Calif. USA) with a thin (˜3 nm) layer of gold-palladium alloy to form a conducting path necessary for electron imaging.

X-Ray Diffraction: A diffractometer (PANalytical X-Pert, Malvern, United Kingdom) was used to perform X-ray diffraction on disc samples from each lot using a scan range of 20° to 70° 2θ with a step size of 0.02° and a time per step of 0.75 s. A Cu K-Alpha source (λ=1.540598 Å) was used with a brass mask (10 mm), a divergence slit (½°), an anti-scatter slit (¼°), and a receiving slit (⅛°).

Contact Angle Measurements: Droplets (25 μL) of deionized water (17.5 MΩ cm resistivity, 75011, Myron L Company, Carlsbad, Calif.) were deposited (VWR Signature Variable Volume Pipette, VWR, Radnor, Pa.) onto sample surfaces (n=3 samples per condition, two measurements per sample) and measured using an optical comparator (2600 Series, S-T Industries, St. James, Minn.) via a procedure established in previous work.

Bacterial Biofilm Assay: An in vitro S. epidermidis (ATCC® 14990™) bacterial biofilm assay was performed per a previously established procedure using n=3 discs per condition. Briefly, Samples were immersed in wells containing 105 cells/mL in a nutrient broth comprised of diluted human plasma and glucose in phosphate buffered saline (PBS). Following exposure (time points of 24 & 48 hours), samples were removed from the broth, rinsed with PBS to remove non-adherent bacteria, and vortexed to separate adherent bacteria from the sample surfaces. Adherent bacteria suspensions were serially diluted and plated onto Petrifilm™ (6400/6406/6442 Aerobic Count Plates, The 3M Company, Minneapolis, Minn.), and colony forming units (CFUs) were counted after a 24 hour incubation period. Differences in CFUs normalized by specimen surface area for each condition were evaluated for statistical significance using a Student's t-test (2-tail, heteroscedastic, 95% confidence) at each time point.

Osteoconductivity Testing: Samples were exposed to KUSA-A1 stem cells in vitro via a previously established procedure for a period of 28 days. Samples were subjected to staining with Alizarin red and subsequent measurement of medium optical density (OD) at 450 nm (n=5), volumetric measurement of deposited mineralized bone tissue (n=10) via confocal scanning laser microscopy (CSLM) (Keyence, Japan), Raman imaging to qualitatively assess formation of bone mineral and soft matrix, and high resolution Raman spectroscopy (n=8) to assess inorganic to organic ratio of the deposited boney tissue, hydroxyapatite (HAp) crystallinity, and ratio of HAp to tricalcium phosphate (TCP).

Flexural Strength: Testing was carried out per 940006—Flexural Strength Test Procedure, which conforms to ASTM C1161-187 (configuration B), on test bars (n=36 bars per lot). Briefly, this is a 3-point bend test with a 40 mm span performed on a universal mechanical test frame (5567, Instron, Norwood, Mass. USA) using a crosshead speed of 0.50 mm/min. Obtained data for each lot were compared using a Student's t-test (2-tail, heteroscedastic, 95% confidence). Further analyses were carried out per ASTM C12399 to obtain Weibull parameters for describing the failure distributions of both material lots. Graphical construction of the Weibull plot was carried out per sections 8.8 and 8.9 of ASTM C1239, and unbiasing of the obtained Weibull moduli were performed per section 9.2 of ASTM C1239. A limited fractographic analysis was conducted using a visible light stereomicroscope (SZX9, Olympus, Japan) for selected low strength (<800 MPa) samples.

Lattice Strain Assessment: Diffraction data were analyzed using commercially available phase analysis software (MATCH!, Crystal Impact, Bonn, Germany) to determine peak locations for [110], [210], [200], and [101] reflections in β-Si₃N₄'s pattern. These are the four principal reflections in the referenced standard pattern (JCPDS-ICDD No. 33-1160) for this material.

### Results and Discussion

Test Disc Characterization: Table 2 shows the dimensional density and linear shrinkage for samples subjected to the various HIP conditions. It can be concluded from the data in Table 2 that the HIPed samples were likely modestly denser than the Sinter Only samples. Further, it appears qualitatively that increasing HIP cooldown pressures yield a modest increase in density.

TABLE 2

| | Dimensional | | |
|---|---|---|---|
| | Density | % Theoretical | S_{L} (diametral) |
| Sample | (g/cm³) | Density | (%) |
| Sinter Only | 3.21 ± 0.01 | 98.23% ± 0.26% | 16.83% ± 0.37% |
| Adiabatic HIP Cool | 3.23 ± 0.04 | 98.66% ± 1.01% | 17.16% ± 0.12% |
| 10 ksi HIP Cool | 3.23 ± 0.02 | 98.88% ± 0.64% | 17.36% ± 0.09% |
| 25 ksi HIP Cool | 3.26 ± 0.02 | 99.70% ± 0.56% | 17.23% ± 0.21% |

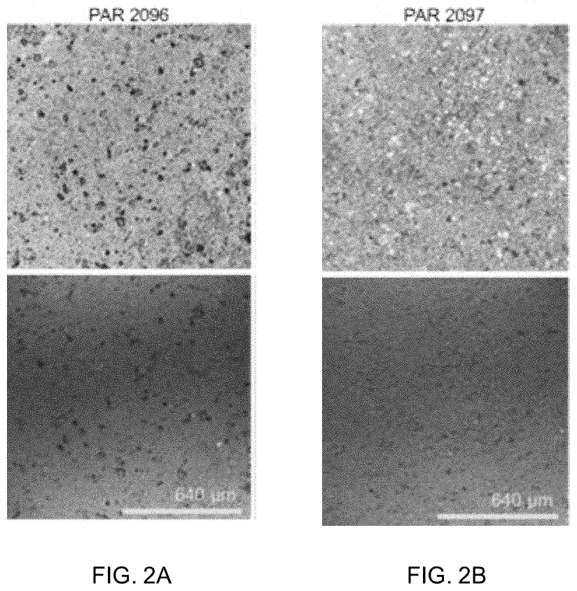

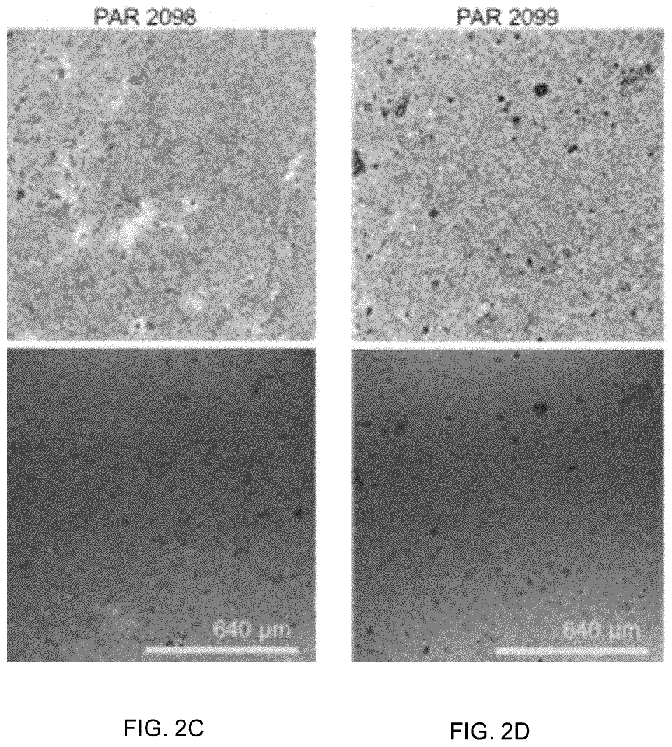

Optical images and corresponding intensity maps captured using CLSM are shown in FIGS. 2A-2D. Corresponding roughness parameter data are presented in Table 3. Defects from pressing, die wall exclusion effects, and hollow agglomerate morphology are apparent. Sa values were similar for all samples at about 1 μm. The lowest absolute measured value was 0.8 μm for the 25 ksi cool sample. While the absolute values for Sa obtained were different (˜2.5 μm) in those measurements, all samples again exhibited similar roughness properties. The disparity in absolute measurement value can be attributed to differences in capture resolution. The data presented here were obtained over ˜2.2 mm² areas using a higher magnification objective (and hence higher resolution/fidelity) while the scans conducted at Piezotech characterized entire disc sample surfaces (˜127 mm²) at lower magnification/resolution in preparation for quantifying volumes of HAp deposited during KUSA-1 MSC experiments.

TABLE 3

| | | | | | Ratio of Actual |
|---|---|---|---|---|---|
| | Sa | Sz | Sv | Sq | Area to |
| Sample | (μm) | (μm) | (μm) | (μm) | Projected Area |
| PAR 2096 | 0.9 | 26.5 | 21.5 | 1.4 | 1.3 |
| No HIP | | | | | |
| PAR 2097 | 1.0 | 29.5 | 21.8 | 1.5 | 1.3 |
| Adiabatic cool | | | | | |
| PAR 2096 | 1.2 | 34.2 | 25.7 | 1.7 | 1.3 |
| 10 ksi cool | | | | | |
| PAR 2096 | 0.8 | 43.6 | 38.0 | 1.5 | 1.3 |
| 25 ksi cool | | | | | |

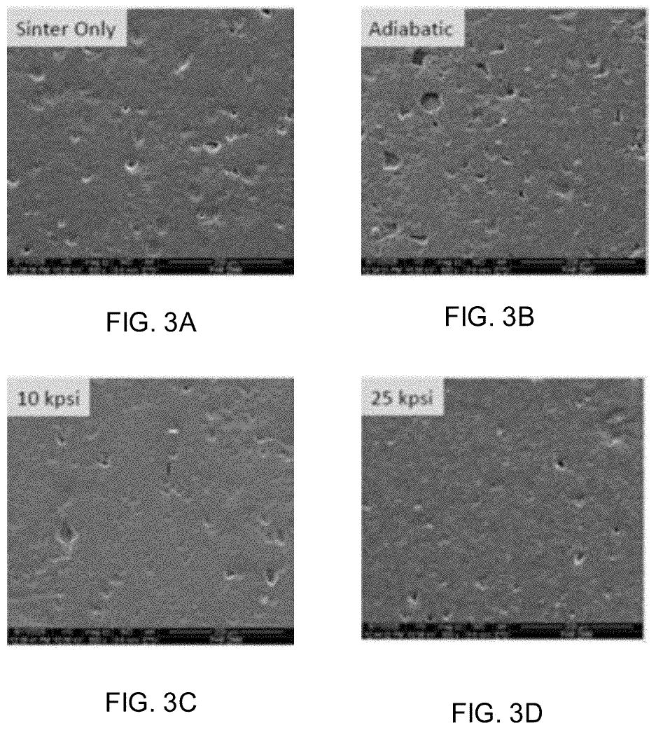

Low magnification secondary electron images in FIGS. 3A-3D show remnant pressing defects from spray-dried agglomerate morphology and die wall exclusion effects. This appears to be most exaggerated in the adiabatic cool sample, and the effect is mitigated as HIP cooldown pressure is held and increased. It is hypothesized that the reduction in defect size is due to a reduction in outgassing of N₂ during the HIP cooldown step. These pressing defect regions are rich in the secondary SiYAlON phase, which melts and fills them during sintering.

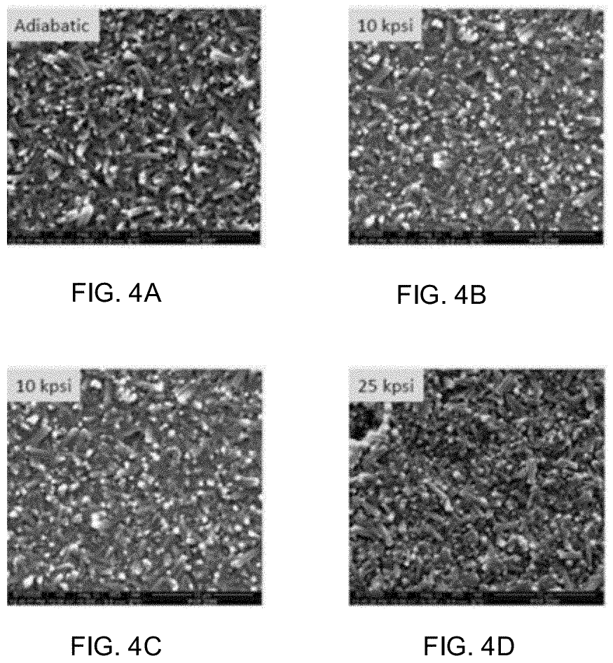

At higher magnification in FIGS. 4A-4D, the classic anisotropic β-phase grain morphology is apparent in all samples. The hexagonal grain cross-sections appear to be somewhat better-defined in the HIP samples relative to the sinter-only sample.

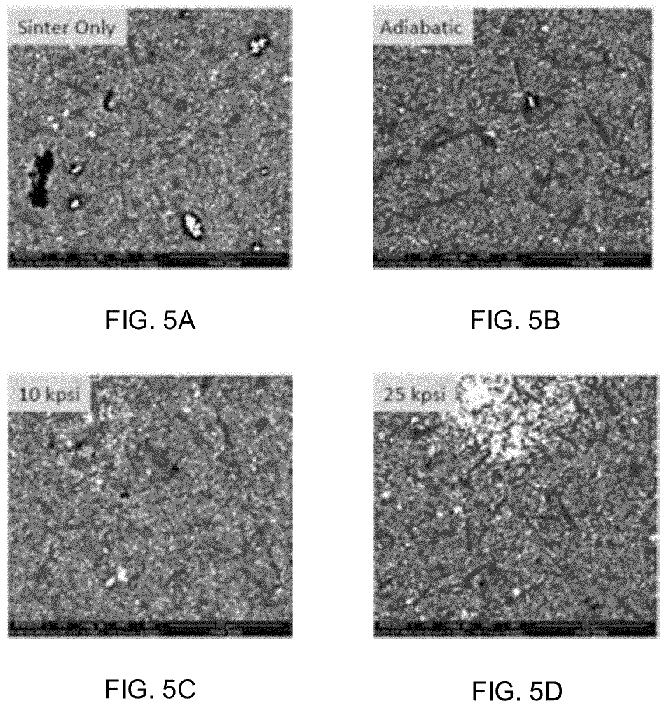

In the back-scatter mode, contrasting by atomic number/density is obtained. This allows for easy visualization of phases with significantly different densities. High density materials reflect more electrons and therefore show up brighter while lower density phases show up darker. In all samples imaged in FIGS. 5A-5D, the classic β-Si₃N₄ microstructure is apparent. Crystalline beta grains show up as dark gray, while the yttria-containing intergranular phase (IGP) shows up much lighter. Finally, the minority colorant phase, thought to be a titanium oxynitride (TiON) solid solution formed due to the inclusion of TiO₂ particles in the Si₃N₄ batch, shows up brightest. Large, closed pores are visible in the sinter only sample along with more agglomerated clusters of the TiON colorant phase relative to the HIP samples. Pores are much finer (˜1 μm) and less prevalent in the adiabatic cool HIP sample. Pores are still present but even finer in the 10 ksi HIP cooldown sample, and the few pores present in the 25 ksi cooldown sample are a small fraction of a micron in size. Crucially, the 25 ksi sample was the only sample where cooldown pressure was raised to a level above the process pressure (22 ksi). It is likely that this greatly mitigated pore formation and growth during cooling. Also of note is that pores seem to form at the interface of IGP and a crystalline grain, lending credence to the assertion that pore formation is a result of precipitation of nitrogen form a supersaturated IGP melt.

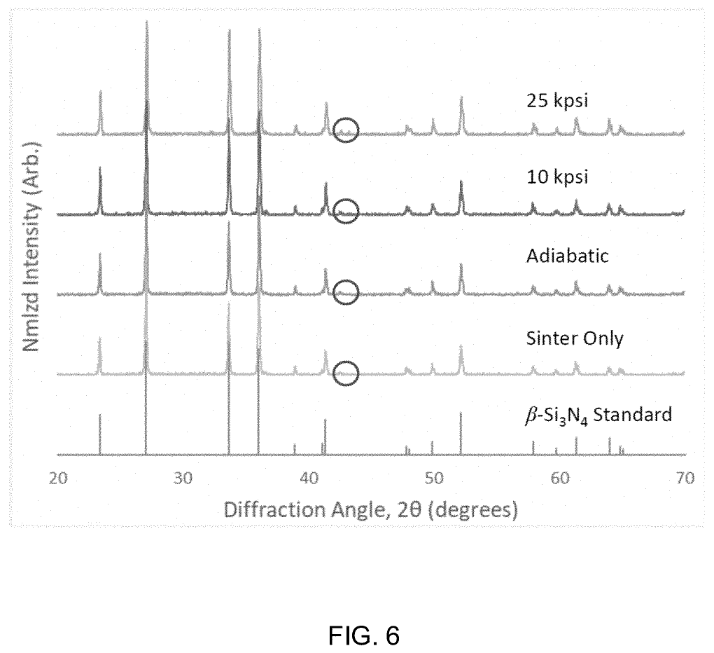

All four sample groups shown in FIG. 6 conform to the β-Si3N4 standard. Some changes in texturing occur from one sample to another. Also, minority peaks in the vicinity of 43° 2θ are not consistent with β-Si₃N₄, but they are consistent with titanium nitride (TiN) (JCPDS #96-110-003511). HIP conditions that increase IGP nitrogen sequestration appear to also drive formation and/or crystallization of the colorant phase formed due to inclusion of TiO₂ in the material batch.

Water contact angles are shown in Table 4. The HIP samples all exhibited very similar water contact angles. The sinter only sample exhibited a significantly larger contact angle. This could have been due to increased surface roughness and porosity relative to the HIP samples. Additionally, the sinter only samples had been stored for a long period while the HIP samples were being processed. As such, there may have been more adventitious carbon buildup on the sinter only samples that was not adequately cleaned away prior to measurement.

TABLE 4

| Sample | Water Contact Angle (°) |
|---|---|
| Sinter Only | 51.8 ± 3.2 |
| Adiabatic HIP | 30.4 ± 7.9 |
| Cool | |
| 10 ksi HIP Cool | 31.3 ± 7.3 |
| 25 ksi HIP Cool | 36.3 ± 5.0 |

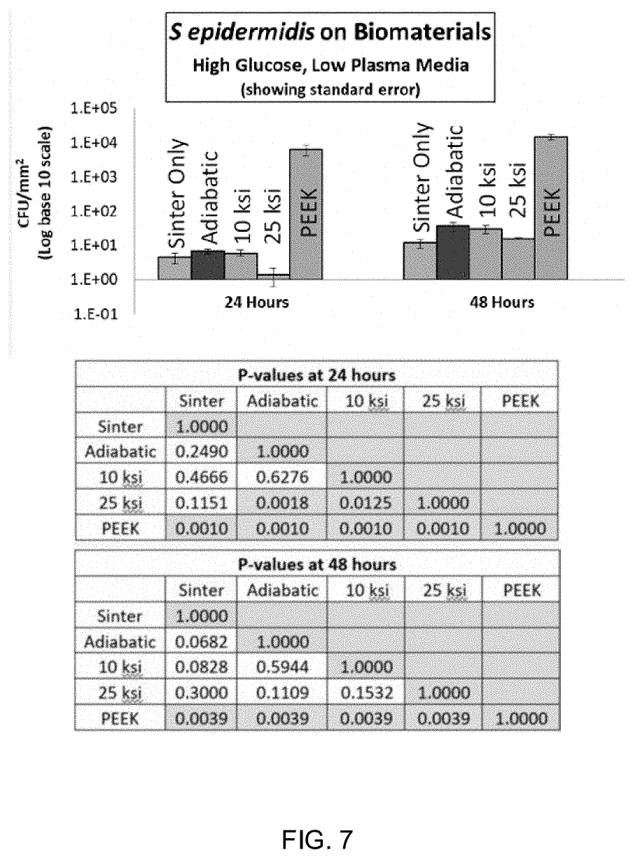

S. epidermidis in vitro Bacterial Biofilm Assay: The data charted in FIG. 7A show a significantly lower CFU/mm² concentration for the 25 ksi HIP cool sample relative to the other HIP samples at the 24 hour time point. However, the difference is less than one logo reduction (See FIG. 7B). At 48 hours, there are no significant differences between any of the silicon nitride samples. Interestingly, at both time points, all silicon nitride samples demonstrate a minimum 2.5 logo reduction relative to the PEEK control, which is a typical result in this assay with this organism. These data suggest that changes in HIP cycle cooldown pressure (or an omitted HIP cycle) did not significantly affect Si₃N₄'s antibacterial properties.

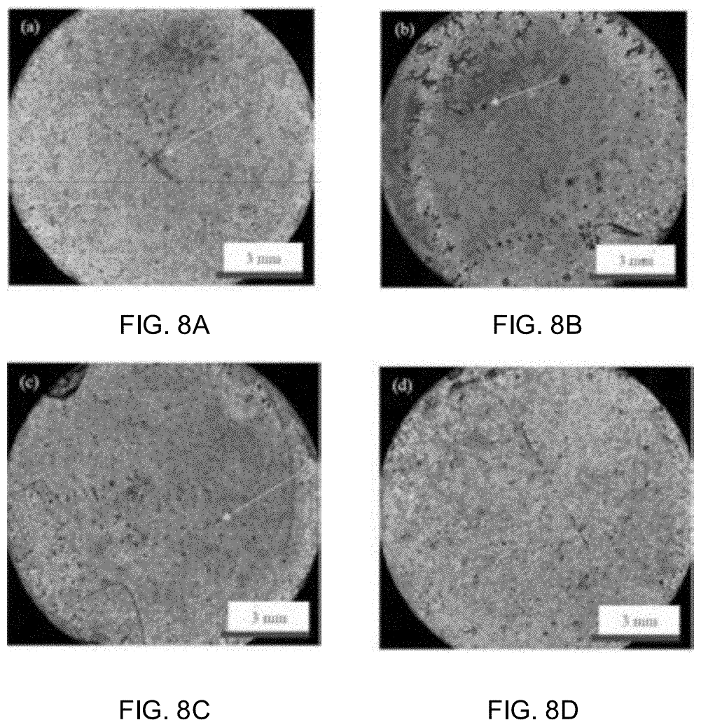

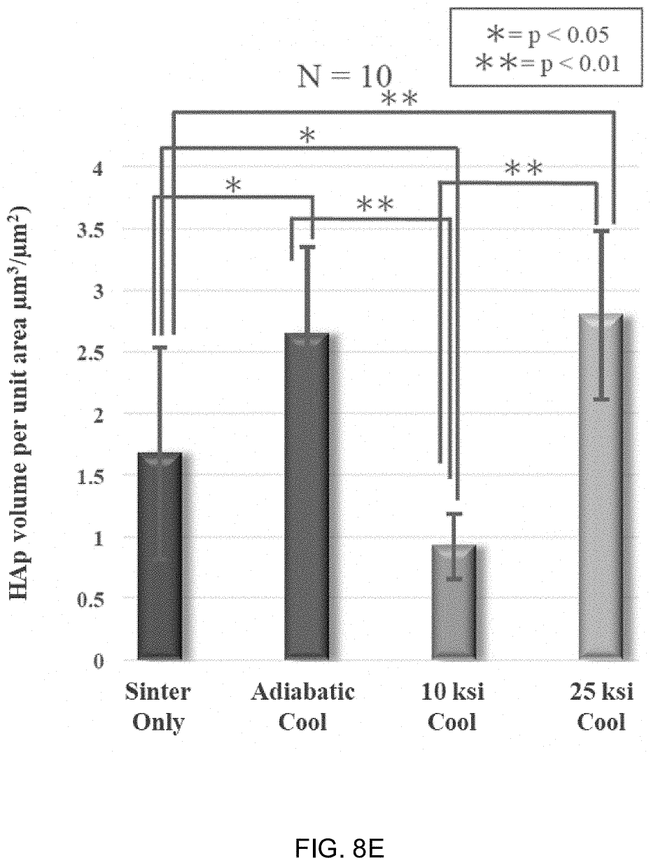

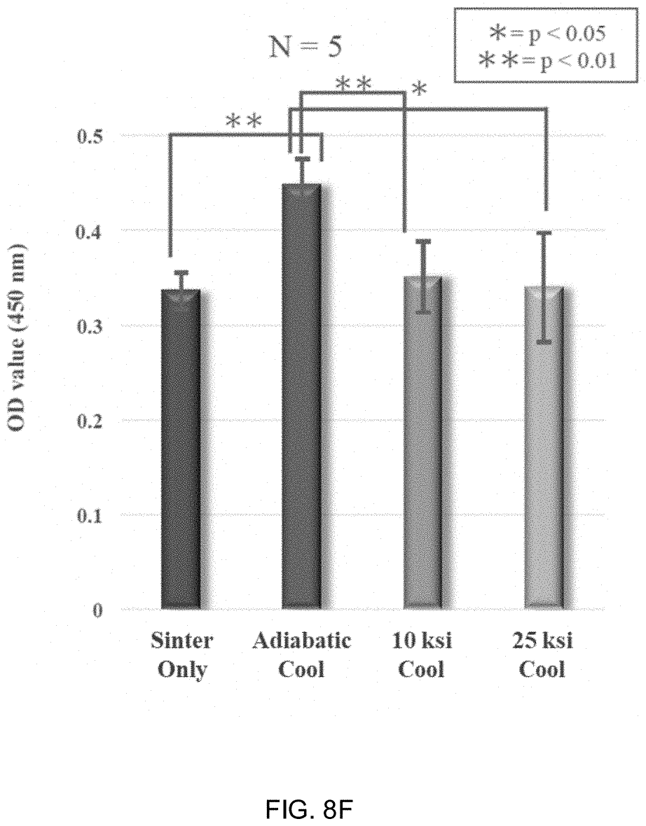

KUSA-A1 MSC Assay 1—Alizarin Red & Volumetric Bone Tissue Deposition: FIGS. 8A-8D show laser scanning micrographs of the silicon nitride samples. Average deposited HAp volume (FIG. 8E) varied from about 1 μm³/μm² for the 10 ksi cool samples to about 2.75 μm³/μm² for the 25 ksi cool samples. The adiabatic cool samples performed comparably to the 25 ksi cool group, and the sinter only group had an intermediate volume of HAp deposited. Excepting the 25 ksi and adiabatic groups, all other samples data sets were statistically significant. However, there was a large deal of variability in these measurements. As an alternate way to assess the presence of mineralized bone tissue, Alizarin red chelates calcium form the mineralized bone tissue to form a complex that absorbs light at 450 nm. A higher optical density value at 450 nm means more calcium has been dissolved form the bone tissue on the samples. Therefore, higher OD₄₅₀ₙₘ means more mineralized bone tissue is present. As can be seen in FIG. 8F, the adiabatic cool sample group exhibited a roughly 30% (statistically significant) higher average OD₄₅₀ₙₘ than the other groups, which yielded results roughly equivalent to one another.

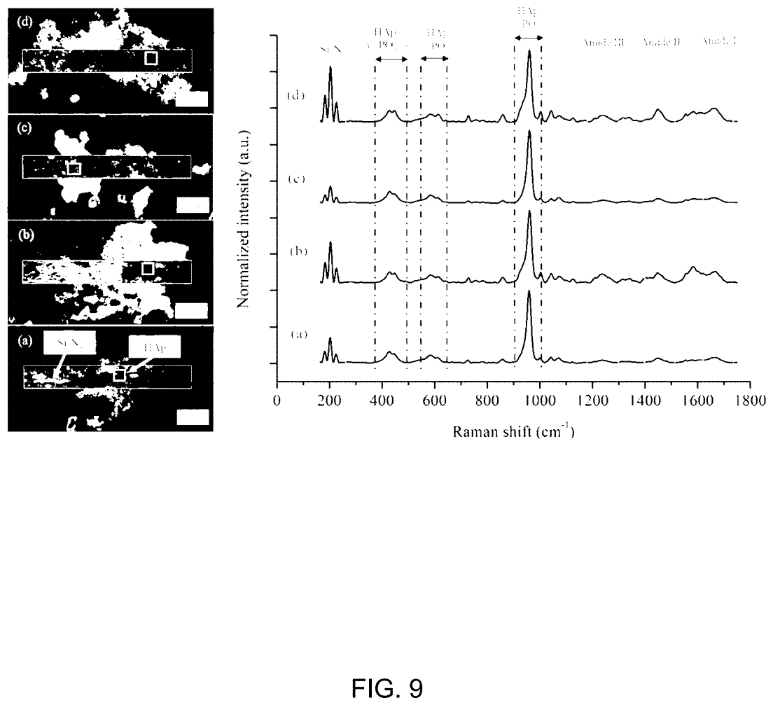

KUSA-A1 MSC Assay 2—Raman Imaging: Raman imaging data presented in FIG. 9 capture spectral features characteristic of bond perturbations in the Si₃N₄ substrate (blue), PO₄³⁻contained in hydroxyapatite and calcium phosphates (red), and amide linkages present in the extracellular polymer (ECP) secreted by the KUSA-1 cells (green). The peaks in the region of 960 cm⁻¹ for the adiabatic and 25 ksi samples are the most well-defined, indicating a higher relative concentration of HAp on those samples. Similarly, the amide peaks are also well-developed for those samples indicating more ECP present. There appears to be little ECP present on the 10 ksi samples, indicating formation of bone tissue that could be embrittled.

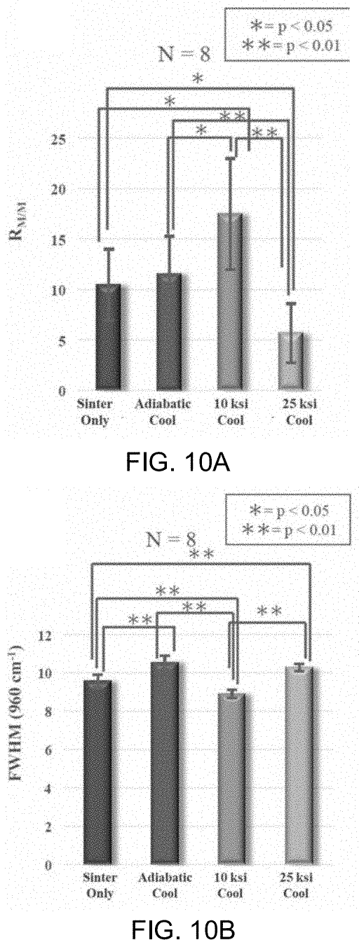

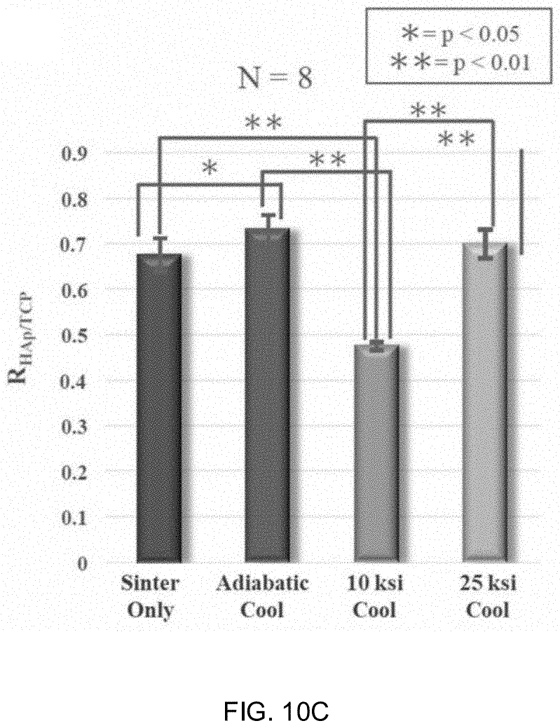

KUSA-A1 MSC Assay 3—High Resolution Raman Spectroscopy: Data in FIG. 10A show that the ratio of mineral or organics in the deposited bone tissue is lowest for the 25 ksi cool condition, highest for the 10 ksi cool condition, and intermediate and roughly equivalent for adiabatic cool and sinter only conditions. However, the only difference where the averages are separated by more than a standard deviation is a comparison between the 10 ksi and 25 ksi cases. The HAp in the bone tissue is more crystalline (see FIG. 10B), and therefore likely a component of more mature bone tissue, in the cases of adiabatic and 25 ksi cool samples (p<0.01). However, the difference across all sample averages is less than 30% of the lowest sample's value. The ratio of hydroxyapatite to tricalcium phosphate (see FIG. 10C), another indicator of bone maturity, is moderately higher for adiabatically cooled samples relative to sinter only and 25 ksi cool samples, which are equivalent to one another. All three of these groups have ratios 70-80% higher than that exhibited for the 10 k ksi group.

Except for the 10 ksi sample group, all groups exhibited similar behavior in terms of amounts and quality of bone tissue produced. The cells exposed to the 25 ksi sample group did appear to secrete more extracellular polymer relative to the others, but this is not necessarily deleterious. The cells on the 10 ksi samples produced the least extracellular polymer, which in turn was less mature and mineralized to a lesser extent than the bone tissue from the other groups. Even though these observed differences were statistically significant, differentiation was still well within an order of magnitude in all cases. Thus, it is doubtful that these moderate differences observed in vitro would translate to significant changes in behavior in vivo.

Flexural Strength: Data presented in Table 5 show the density and average strength of silicon nitride produced using the 25 ksi HIP cooldown cycle.

TABLE 5

| | Density | Average σ₃ₚ | Student's t-test |
|---|---|---|---|
| Material | (g/cm³) | (MPa) | p-value |
| Adiabatic HIP cool | 3.257 ± 0.003 | 1000 ± 126 | p << 0.05 |
| (PAR 2059) | | | (~1 × 10⁻¹³) |
| 25 ksi HIP cool | 3.258 ± 0.004 | 1253 ± 104 | |
| (PAR 2047) | | | |

Measured density is equivalent to material produced using the standard production adiabatic cooling cycle. The weakest bars in the adiabatic cool lot failed at strengths between 650 and 700 MPa, indicating defect-driven behavior. Notably, the weakest bar in the 25 ksi cool lot failed at 1000 MPa, which was the average strength for the adiabatic cool lot. The strongest bar from the adiabatic cool lot failed at 1170 MPa, while the strongest bar from the experimental lot failed at 1420 MPa. Average flexural strength for the experimental lot is increased by about 250 MPa, and a Student's t-test (2-tail, heteroscedastic) shows that the differences between the two flexural experiment sample sets are statistically significant. The differences between the lot average, maximum, and minimum strength indicate a shift on the order of 200-300 MPa in the entire strength distribution between the two groups. Generally, defect eliminations tend remove low strength tails from strength distributions without affecting the strength values at the less defect-affected high end of the distribution. So, the complete distribution shift could be indicative of either removal/size reduction of an entire flaw population or some other fundamental material change occurring.

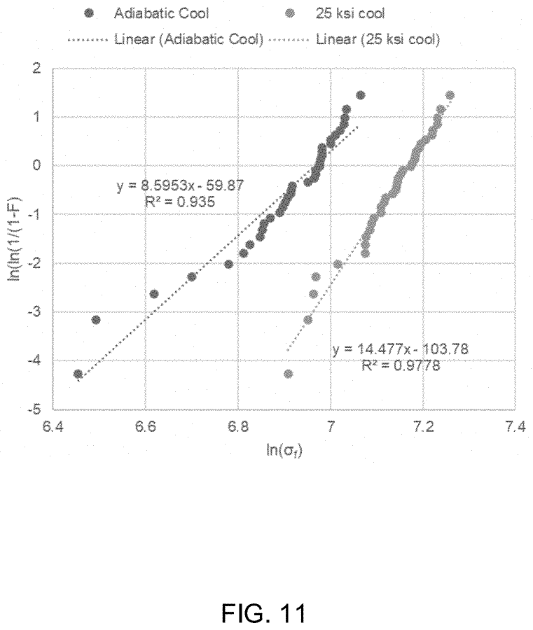

Brittle material failure distributions tend to better conform to the Weibull distribution than the normal distribution. Therefore, the failure data for both lots are presented as a Weibull probability plot in FIG. 11. The adiabatic cool lot exhibits a characteristic strength of 1059 MPa and a Weibull modulus of 8.60. The 25 ksi cool lot exhibits a characteristic strength of 1298 MPa and a Weibull modulus of 14.48. Per ASTM C1239, the unbiasing factor for Weibull modulus derived from lot data with n=36 test specimens is 0.962. Therefore, the unbiased Weibull modulus values are 8.27 and 13.93 for the adiabatic cool lot and 25 ksi cool lot, respectively. This shift in characteristic strength and modulus represents a significant improvement in lot characteristics. Both lots conform reasonably well to the Weibull distribution (R²=0.935 for adiabatic cool and 0.9778 for 25 ksi cool), but a few low strength failures in the adiabatic cool group deviate relatively far from the linear fit. These failures were likely caused by flaws in the material. The chart also shows very clearly the shift in the behavior of the entire distribution rather than only elimination of low strength defectives.

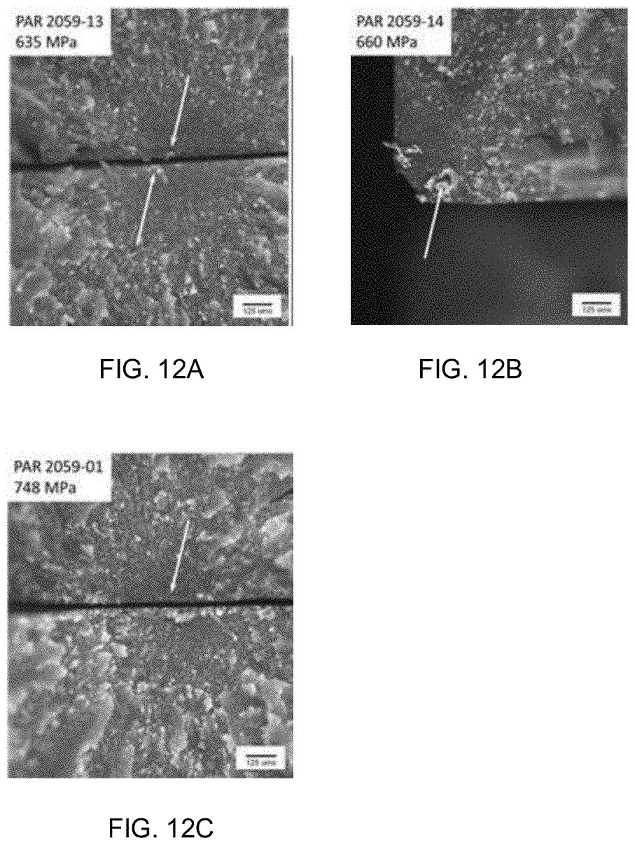

Images of fracture origins for specimens that failed below 800 MPa are presented in FIG. 12. Since the lowest strength bar in the 25 ksi HIP cool lot failed at 1000 MPa, all imaged specimens are from the adiabatic HIP cool lot with failure strengths in the range of 635-748 MPa. Classic mirror-mist-hackle patterns are observed in all cases. Specimens 2059-01 and -13 have inclusions readily apparent at their fracture origins. Specimen 2059-14 appears to have an inclusion at the origin but also looks as if it could have been edge-loaded. The inclusions in specimens -01 and -13 are consistent with (dark, highly-reflective) glass-rich defects that form as a result of green state flaws filling with SiYAlON IGP during processing.

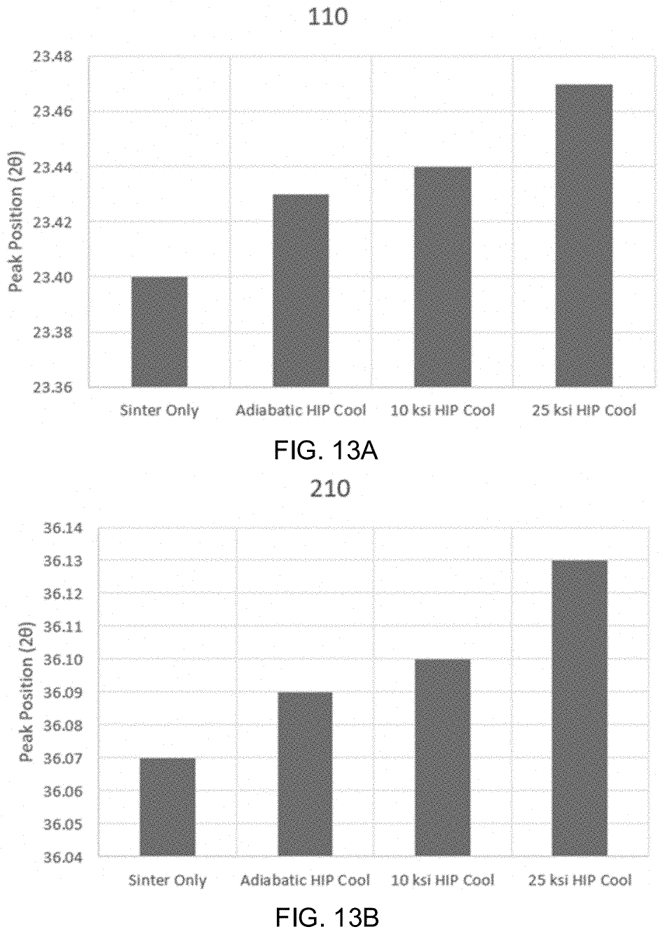

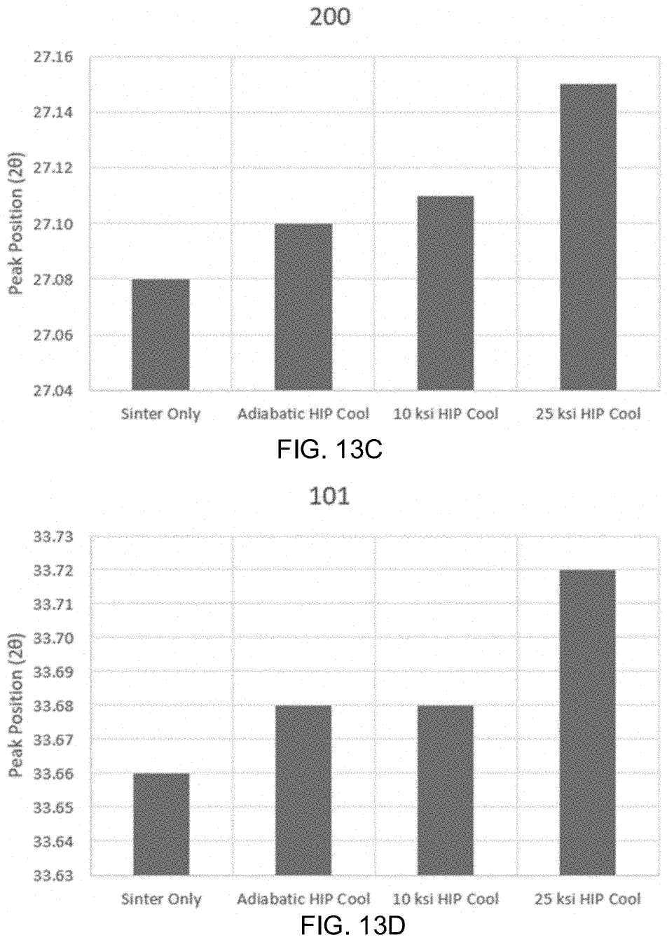

Lattice strain from XRD peak shift: Charts showing the peak positions for the four primary reflections for β-Si₃N₄ are presented in FIGS. 13A-13D. In all cases, the peak location shifts to a higher angle by a similar magnitude as higher nitrogen overpressure is applied during cooldown from the final firing cycle.

Eqn. 1 is Bragg's Law, where n is the order of reflection, λ is the wavelength of the incident radiation, d is the interplanar spacing within the crystal, and θ is the angle of incidence.

$$n λ = 2 d sin θ$$

Since nλ is constant for a given reflection and radiation source, and sine increases as θ increases up to θ=90°, a peak shift to a higher diffraction angle in the observed range corresponds to a narrower d-spacing between atomic planes of that peak's orientation. Observation of the same trend in peak shift with very similar magnitudes of diffraction angle increase for all principal peaks is consistent with β-phase crystallites in the HIP samples being in a state of residual isotropic compressive strain whose magnitude increases as applied pressure during HIP cooldown increases. This is also consistent with a higher overpressure causing a higher degree of nitrogen supersaturation in the continuous glass phase resulting in (1) closure of volumetric defects (2) prevention of pore formation from N₂ evolution, and (3) residual strain volumetric constraint.

### Conclusions

Controlling the cooldown phase of the HIP cycle has significant impact on the resulting Si₃N₄ material's microstructure and properties. Holding the gas pressure constant during cooling mitigates evolution of nitrogen at intermediate temperatures where the solubility of nitrogen in the glass decreases but mobility is still relatively high. This reduces the size of pores that form during the cooldown phase of the HIP process. Holding the pressure constant at a value greater than the process pressure appears to greatly reduce the size and frequency of pores in the microstructure. Decreasing the size and frequency of these pore defects leads to a direct improvement in strength as expected from well-established fracture mechanics science. Further, XRD data show that a residual stress state exists in the material that is proportional to the magnitude to the pressure applied during HIP cooldown. This residual stress state could contribute to the large observed improvement in strength that accompanies processing with a high pressure hold during HIP cooldown.

No deleterious effects were observed for bacterial biofilm assay performance or osteoconductivity test performance for the material subjected to the 25 ksi HIP cooldown cycle, and the strength increase that could be realized is substantial. The experiments that showed a large improvement in strength and reliability should be repeated as part a verification effort component of a formal controlled process change to introduce the 25 ksi cooldown HIP cycle into MC² processing.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for manufacturing a component comprising a ceramic or glass material, the method comprising:
receiving the ceramic or glass material within a containment vessel;
simultaneously heating and applying isostatic pressure to the ceramic or glass material within the containment vessel to a first temperature and a first pressure using a pressurizing nitrogen gas;
holding the first temperature and the first pressure for a period of time;
cooling the ceramic or glass material within the containment vessel to a second temperature while maintaining the first pressure; and
depressurizing the containment vessel to a second pressure, wherein the component comprises the ceramic or glass material supersaturated with nitrogen.

2. The method of claim 1, further comprising removing the component from the containment vessel.

3. The method of claim 1, wherein the ceramic or glass material is a powder.

4. The method of claim 1, wherein the ceramic or glass material is sintered prior to being received within the containment vessel.

5. The method of claim 1, wherein the ceramic or glass material is pre-formed prior to being received within the containment vessel.

6. The method of claim 1, wherein the ceramic material is silicon nitride.

7. The method of claim 1, wherein the first temperature is about 1400° C. to about 1800° C.

8. The method of claim 1, wherein the first pressure is about 150 MPa to about 300 MPa.

9. The method of claim 1, wherein the period of time is about 0.5 hours to about 2 hours.

10. The method of claim 1, wherein the second temperature is about 25° C. to about 200° C.

11. The method of claim 1, wherein the second pressure is about atmospheric pressure.

12. The method of claim 1, wherein the ceramic or glass material in the component is supersaturated with about 10% to about 15% nitrogen.

13. The method of claim 1, wherein the ceramic or glass material further comprises about 0.1 wt. % or more of sodium oxide (Na₂O), lithium oxide (Li₂O), potassium oxide (K₂O) magnesium oxide (MgO), aluminum oxide (Al₂O₃), yttrium oxide (Y₂O₃), ytterbium oxide (Yb₂O₃), lanthanum oxide (La₂O₃), strontium oxide (SrO), calcium oxide (CaO), silicon dioxide (SiO₂), zirconium oxide (ZrO₂), boron trioxide (B₂O₃), phosphorus pentoxide (P₂O₅), or combinations thereof.

14. The method of claim 1, wherein the cooling step increases an average flexural strength of the component by 200-300 MPa as compared to a component produced using adiabatic cooling.

15. An implant comprising a ceramic or glass material supersaturated with nitrogen, wherein the implant is produced by a method comprising:
receiving the ceramic or glass material within a containment vessel;
simultaneously heating and applying isostatic pressure to the ceramic or glass material within the containment vessel to a first temperature and a first pressure using pressurizing nitrogen gas;
holding the first temperature and the first pressure for a period of time;
cooling the ceramic or glass material within the containment vessel to a second temperature while maintaining the first pressure; and
depressurizing the containment vessel to a second pressure.

16. The implant produced by the method of claim 15, wherein the ceramic material is silicon nitride.

17. The implant produced by the method of claim 15, wherein the ceramic or glass material in the component is supersaturated with about 10% to about 15% nitrogen.

18. The implant produced by the method of claim 15, wherein the implant further comprises about 0.1 wt. % or more of sodium oxide (Na₂O), lithium oxide (Li₂O), potassium oxide (K₂O) magnesium oxide (MgO), aluminum oxide (Al₂O₃), yttrium oxide (Y₂O₃), ytterbium oxide (Yb₂O₃), lanthanum oxide (La₂O₃), strontium oxide (SrO), calcium oxide (CaO), silicon dioxide (SiO₂), zirconium oxide (ZrO₂), boron trioxide (B₂O₃), phosphorus pentoxide (P₂O₅), or combinations thereof.

19. The implant produced by the method of claim 15, wherein the implant is antipathogenic.

20. The implant produced by the method of claim 15, wherein the implant inhibits the proliferation of at least one of bacteria, fungi, and viruses.

21. The implant produced by the method of claim 15, wherein the implant has an average flexural strength that is 200-300 MPa higher than an implant produced using adiabatic cooling.
